(19) United States
(12) Patent Application Publication
(10) Pub. No.: US 2011/0098196 A1
(43) Pub. Date: Apr. 28, 2011

(54) Multiplex Screening for Pathogenic Hypertrophic Cardiomyopathy Mutations

(75) Inventors: James J. Dermody, Boonton, NJ (US); Marvin Schwalb, Livingston, NJ (US); Peter Tolias, Westfield, NJ (US)

(73) Assignee: UNIVERSITY OF MEDICINE AND DENTISTRY OF NEW JERSEY, Somerset, NJ (US)

(21) Appl. No.: 12/934,509

(22) PCT Filed: Mar. 25, 2009
(86) PCT No.: PCT/US09/38204

§ 371 Date: Dec. 13, 2010

Related U.S. Application Data
(60) Provisional application No. 61/070,794, filed on Mar. 25, 2008.

(51) Int. Cl. C40B 30/00; C12Q 1/68; C40B 30/04; C12M 1/34; C40B 60/12

(52) U.S. Cl. 506/9; 435/6; 506/7; 435/287.2; 506/39

(57) ABSTRACT

This invention relates to a new method of screening for hypertrophic cardiomyopathy. In certain embodiments, the invention comprises a method of screening for hypertrophic cardiomyopathy comprising detecting the presence or absence of at least one pathogenic HCM mutation by mutation detection assay in a sample from a subject to be tested for hypertrophic cardiomyopathy.

### CLAIM OF PRIORITY

This application claims priority to U.S. Provisional Application No. 61/070,794 filed Mar. 25, 2008, the disclosure of which is hereby incorporated by reference in its entirety.

### BACKGROUND OF THE INVENTION

Hypertrophic cardiomyopathy (“HCM”) is an often fatal but manageable disease. The incidence is reported to be about 1/400 (approximately 750,000) in the general U.S. population. The variable expressivity of this disease suggests it may be higher, making HCM the most common monogenic cardiac disorder in the U.S. Macon and McKenna et al., ACC/ESC Expert Consensus Document on Hypertrophic Cardiomyopathy, J of American College of Cardiology (2003) 42: 1-27. In addition, it is the most frequent cause of unexpected sudden death in teenagers and young adults. Elliott, Poloniecki et al., Sudden death in hypertrophic cardiomyopathy: Identification of high risk patients, J of American College of Cardiology (2000) 36: 2212-2218. The disease is characterized by a thickening of the heart muscle (hypertrophy) in the absence of hypertension or any other apparent cause. HCM is difficult to diagnose. Clinical presentation and progression of HCM varies widely among affected patients and the symptoms (breathlessness especially during exercise, heart palpitations, dizziness and fainting) are common to many other conditions. The most common misdiagnosis is asthma, specifically athletically induced asthma, likely due to the shortness of breath often observed in many HCM patients. It is also common for HCM patients to be initially diagnosed with anxiety attacks, panic attacks, or some form of depression only to later discover the cause of the patient's symptoms is HCM. An echocardiogram is typically used to help establish a clinical diagnosis, but there remains a need for more facile methods of diagnosis that do not rely on the often misleading observation of symptoms.

Researchers and clinicians have also established that HCM has a strong genetic component, as the disease tends to run in families. Approximately half of the clinically diagnosed HCM cases are associated with dominant mutations in genes that specify components of the heart's contractile machinery. In fact, in 2006 the American College of Cardiology and the American Heart Association released joint guidelines (“Guidelines for the Management of Patients with Ventricular Arrhythmias and the Prevention of Sudden Cardiac Death”) that recommend genetic testing for patients suspected of having HCM.

Genetic studies have confirmed HCM as a disease of the contractile proteins in heart muscle cells caused by mutations in 11 genes: 1) beta-cardiac myosin heavy chain; 2) cardiac myosin-binding protein C; 3) cardiac troponin-T; 4) cardiac troponin-I; 5) alpha-tropomyosin; 6) cardiac essential myosin light chain; 7) cardiac regulatory myosin light chains; 8) actin; 9) alpha-myosin heavy chain; 10) titin; and 11) muscle LIM protein. To date, genetic testing for HCM has consisted of complete DNA sequencing of 6 or more of these 11 genes at a cost of several thousand dollars, which results in a very expensive and time-consuming diagnostic process and has precluded a majority of patients from accessing this important resource. This high cost has been a major impediment to genetic diagnosis. The benefit of having access to an affordable genetic test for patients suspected of having HCM is clear in that early intervention and treatment, including implanting of defibrillators, can save lives. Also, many family members of HCM patients would be highly motivated to participate in testing since the parents, siblings, and children of an individual with a HCM mutation have a 50% risk of having the same mutation and are thus at high risk for HCM themselves. Therefore, testing for HCM mutations in a patient's family members is quite beneficial. There remains a need for cost effective HCM diagnosis, especially for individuals with a family history and for those at high risk of sudden death.

By 2006, 434 mutations (listed at http://genetics.med.harvard.edu/˜seidman/cg3) were revealed by traditional DNA sequencing of genetic material from HCM patients, and this number is expected to increase as research continues in the field. However, the clinical utility of any particular mutation is not obvious. Numerous benign DNA mutations (polymorphisms) are known as well as disease causing (pathogenic) mutations and, objective criteria to distinguish between the two are required. The national Hypertrophic Cardiomyopathy Association (the “HCMA”) is currently compiling a nationwide database of known HCM-related mutations. As the number of known HCM mutations increases, it is essential that we identify the subset that are pathogenic and interrogate them using efficient and affordable multiplexing diagnostic methods. Thus, for widespread molecular screening, there exists an unmet medical need for more conclusive and cost-efficient methods of HCM diagnosis.

All references cited in this application are hereby incorporated by reference in their entireties.

### SUMMARY OF THE INVENTION

The instant invention relates to a new method of testing for hypertrophic cardiomyopathy (HCM).

The method of the instant invention establishes criteria for defining mutations as pathogenic. For the purposes of this application, a mutation is “pathogenic” if it falls under at least one of the following categories:
Category 1: It is predicted to cause an amino acid substitution (missense mutation) and is present in two or more clinically diagnosed HCM patients.
Category 2: Its predicted consequence is the absence of the encoded protein (i.e. nonsense mutations, insertions or deletions causing a protein frame shift, and sequence changes that affect RNA splicing).

For the purposes of this application, “mutation detection” means any method known in the art whereby particular pathogenic HCM mutations of interest are screened for within a single or small series of multiplexed assays, as opposed to the traditional genetic sequencing methods whereby entire genomic regions are sequenced in full. In some embodiments, mutation detection comprises detection of mutations by hybridization with sequence-specific oligonucleotide probes. In other embodiments, mutation detection comprises selective amplification of specific alleles. In further embodiments, mutation detection comprises detection of sequence variation using primer extension.

In some embodiments, mutation detection comprises a solid-phase, particle-based allele specific mutation detection assay such as the ILLUMINA® VeraCode BeadXpress multiplex platform or the LUMINEX® xTAG multiplex platform (which has an install base of over 5,000 units across the U.S. in both research and clinical diagnostic settings) for multiple mutation detection.

In certain embodiments, the invention comprises a method of screening for hypertrophic cardiomyopathy comprising detecting the presence or absence of at least one pathogenic HCM mutation by mutation detection assay in a sample from a subject to be tested for hypertrophic cardiomyopathy.

In further embodiments, the invention comprises a method of screening for hypertrophic cardiomyopathy comprising detecting the presence or absence of at least one pathogenic HCM mutation by particle based allele specific mutation detection assay in a sample from a subject to be tested for hypertrophic cardiomyopathy, wherein for each HCM mutation to be detected, the assay utilizes one oligonucleotide that matches the mutant DNA sequence and one oligonucleotide that matches the corresponding normal sequence; and each oligonucleotide contains specific sequences that match complementary oligonucleotide sequences on the individual detection particles.

In certain embodiments, the detection is performed by multiplex assay.

Certain embodiments of the present invention comprise a panel of at least 10 pathogenic mutations, according to the methods described herein. In other embodiments, the present invention comprises a panel of at least 100 pathogenic mutations. In further embodiments, the present invention comprises a panel of at least 150 pathogenic mutations. In certain embodiments, the present invention comprises a panel of from 50 to 600 pathogenic mutations. In other embodiments, the present invention comprises a panel of from 100 to 500 pathogenic mutations. In further embodiments, the present invention comprises a panel of from 50 to 300 pathogenic mutations. In other embodiments, the present invention comprises a panel of from 200 to 500 pathogenic mutations.

In some embodiments, the catch rate of the method which identifies pathogenic mutations in the HCM associated genes is at least 40%. In further embodiments, the catch rate of the method is at least 60%. In further embodiments, the catch rate of the method is at least 80%. In still further embodiments, the catch rate of the method is at least 95%. In other embodiments, the catch rater of the method is from 40% to 80%. In further embodiments, the catch rate of the method is from 40% to 95%. In other embodiments, the catch rater of the method is from 40% to 70%.

In certain embodiments of the invention, the detection is performed by particle based allele specific mutation detection.

In certain embodiments, the invention comprises detecting the presence or absence of at least one mutation that is predicted to cause an amino acid substitution and is present in two or more clinically diagnosed HCM patients. In other embodiments, the invention comprises detecting the presence or absence of at least one mutation whose predicted consequence is the absence of an encoded protein.

In certain embodiments, the invention comprises detecting the presence or absence of at least one mutation (appearing in Richard et. al. (2003) Circulation 107: 2227-2232) selected from those set forth on Tables 1 and 2, e.g. A6491G, G6643A, T6685C, G8278A, G8848T, G8848A, C8847T, C9123T, A9483G, G10457A, G11282A, G12138A, C12307T, G12361A, delE930, C19222T, AND C19236T in beta-cardiac Myosin Heavy Chain; A5254C, G5256A, G7360A, G11070C, A15829G, G17721A, G20410T, del2376-2381, G5828A, A7308G, A10385G, del10512-10513, delT10587, delC10618, del11047-11048, T11073C, delA12413, A13858G, dup15042-15063, G15131A, A15829G, insG15919, del16189-16193, del16190-16194, delC16212, del17773-17774, del18566-18567, delG21059, ins21404-21415, and del21420-21423 in cardiac Myosin-Binding Protein C; F70L, R102L, P120V, N271I, and W287ter in cardiac Troponin T; and F18L, R58Q, and aIVS5g in cardiac Regulatory Myosin Light Chain. In further embodiments, the invention comprises detecting the presence or absence of at least 10 mutations selected from Tables 1 and 2. In other embodiments, the invention comprises detecting the presence or absence of at least 20 mutations selected from Tables 1 and 2. In other embodiments, the invention comprises detecting the presence or absence of at least 30 mutations selected from Tables 1 and 2. In other embodiments, the invention comprises detecting the presence or absence of at least 40 mutations selected from Tables 1 and 2. In other embodiments, the invention comprises detecting the presence or absence of at least 50 mutations selected from Tables 1 and 2.

In certain embodiments, the invention comprises a method of diagnosing hypertrophic cardiomyopathy comprising detecting the presence of at least one pathogenic HCM mutation by mutation detection assay in a sample from a subject to be tested for hypertrophic cardiomyopathy.

In certain embodiments, the invention comprises a method of diagnosing hypertrophic cardiomyopathy comprising detecting the presence of at least one pathogenic HCM mutation by a particle based allele specific mutation detection assay in a sample from a subject to be tested for hypertrophic cardiomyopathy, wherein for each HCM mutation to be detected, the assay utilizes one oligonucleotide that matches the mutant DNA sequence and one oligonucleotide that matches the corresponding normal sequence; and each oligonucleotide contains specific sequences that match complementary oligonucleotide sequences on individual detection particles.

The sample of the instant invention may be any body fluid and/or tissue from which DNA can be obtained by means known to those in the art. In preferred embodiments, the sample comprises cheek cells. In other embodiments, the sample comprises a blood, sputum or skin sample.

Certain embodiments of the present invention comprise a diagnostic apparatus comprising a mutation detection system capable of detecting the presence or absence of at least one pathogenic HCM mutation in a sample from a subject to be tested for hypertrophic cardiomyopathy.

Further embodiments of the instant invention comprise a diagnostic apparatus comprising a mutation detection system capable of detecting the presence or absence of at least one pathogenic HCM mutation by particle based allele specific mutation detection assay in a sample from a subject to be tested for hypertrophic cardiomyopathy, wherein for each HCM mutation to be detected, the assay utilizes one oligonucleotide that matches the mutant DNA sequence and one oligonucleotide that matches the corresponding normal sequence; and each oligonucleotide contains specific sequences that match complementary oligonucleotide sequences on the individual detection particles.

### DETAILED DESCRIPTION OF THE INVENTION

It has recently been found that HCM is an excellent candidate for diagnostic testing by direct mutation detection analysis. The instant invention establishes criteria for defining specific HCM mutations in key HCM genes as “pathogenic” and combines those pathogenic mutations into a single affordable mutation detection test. This combination of screening for only pathogenic mutations via direct mutation detection analysis yields more definitive results in a more cost-efficient manner.

Current diagnostic tests for HCM typically consist of complete DNA sequencing of 6-11 genes and do not test specifically for the presence or absence of particular mutations. These DNA sequencing methods have the drawback of revealing any and all DNA mutations in the genes tested, including non-pathogenic polymorphic variants. Further, some HCM mutations are known in genes that are not part of the standard DNA sequencing panel. Whereas a new genetic variant identified from DNA sequencing analysis is by no means conclusive, a positive result for a pathogenic mutation identified in a detection test provides definitive results in most HCM patients while also revealing an inexpensive specific mutation test that can be offered to high risk family members of the diagnosis subject.

Performing diagnosis with HCM mutations that have been deemed “pathogenic” is also expected to improve the “catch rate” of diagnostic tests.

Therefore, one embodiment of the instant invention comprises a unique single or set of panels on the ILLUMINA or LUMINEX® platforms. The panels consists of up to all identified pathogenic mutations of the 434 known mutations previously identified by DNA sequencing.

Another embodiment of the present invention comprises a panel of up to 55 mutations that have been deemed pathogenic according to the criteria set forth in Category 1 and Category 2 above (see also Table 1 and Table 2).

Once a mutation has been identified in a patient, that individual's first-degree relatives (siblings, children and parents) all share a 50% risk of having the same mutation. Since early detection of HCM dramatically improves its clinical management, and alerts pre-symptomatic mutation carriers to the significant risk of sudden death, such family members will be highly motivated to seek genetic testing and will be able to do so with the less expensive option provided by the instant invention.

In preferred embodiments of the instant invention, DNA from cheek cells harvested on a cytology brush is utilized, although blood, skin, or any other tissue sample or body fluid can be also used. The existing tests require a 5-7 cc blood sample. Patients find the cheek cell analysis a more convenient and less painful method of sample collection and precludes the need for a doctor's visit to draw blood.

An important aspect of this assay is that it takes into account the detection of mutations that are most likely to be pathogenic, e.g. (a) mutations that have been predicted to cause an amino acid substitution and are present in two or more clinically diagnosed HCM patients and/or (b) the mutation's predicted consequence is the absence of the encoded protein, as set forth in Category 1 and Category 2 above. This is an advantage over the genetic tests that are currently in use, most especially because it can detect mutations in genes that are not included in the current DNA sequencing assays.

Once a diagnosis of HCM is made, the patient can be treated according to general norms as are known in the art.

TABLE 1
Mutations (appearing in Richard et. al. (2003)
Circulation 107: 2227-2232) fulfilling the defined
pathogenic criteria defined in Category 1

| | cardiac | | cardiac |
|---|---|---|---|
| beta-cardiac | Myosin- | | Regulatory |
| Myosin Heavy | Binding | cardiac | Myosin Light |
| Chain | Protein C | Troponin T | Chain |
| A6491G | A5254C | F70L | F18L |
| G6643A | G5256A | R102L | R58Q |
| T6685C | G7360A | P120V | |
| G8278A | G11070C | N271I | |
| G8848T | A15829G | | |
| G8848A | G17721A | | |
| C8847T | G20410T | | |
| C9123T | | | |
| A9483G | | | |
| G10457A | | | |
| G11282A | | | |
| G12138A | | | |
| C12307T | | | |
| G12361A | | | |
| delE930 | | | |
| C19222T | | | |
| C19236T | | | |

TABLE 2
Mutations (appearing in Richard et. al. (2003)
Circulation 107: 2227-2232) fulfilling the defined
pathogenic criteria defined in Category 2

| cardiac Myosin-Binding | | cardiac Regulatory |
|---|---|---|
| Protein C | cardiac Troponin T | Myosin Light Chain |
| del2376-2381 | W287ter | aIVS5g |
| G5828A | | |
| A7308G | | |
| A10385G | | |
| del10512-10513 | | |
| delT10587 | | |
| delC10618 | | |
| del11047-11048 | | |
| T11073C | | |
| delA12413 | | |
| A13858G | | |
| dup15042-15063 | | |
| G15131A | | |
| A15829G | | |
| insG15919 | | |
| del16189-16193 | | |
| del16190-16194 | | |
| delC16212 | | |
| del17773-17774 | | |
| del18566-18567 | | |
| delG21059 | | |
| ins21404-21415 | | |
| de21420-21423 | | |

### EXAMPLES

### Allele Specific Primer Extension (“ASPE”) Reactions and Bead Based Allele Specific Mutation Detection

These methods are standard for LUMINEX® bead-based mutation detection.

For any LUMINEX® based assay, two allele specific oligonucleotides are needed for each mutation, one oligonucleotide that matches the mutant DNA sequence and one oligonucleotide that matches the corresponding normal sequence. In addition, these oligonucleotides are synthesized with specific “Tag” sequences that will match complementary oligonucleotide “Tag” sequences on individual detection beads.

The ASPE oligonucleotides serve as primers for an extension reaction driven by DNA polymerase, which includes biotin-dCTP as a colorimetric measure of allele specific DNA synthesis, so that primer extension only occurs when the DNA synthesis complex forms on a perfectly matched primer-template combination. Biotin-labeled extension products are hybridized to bead immobilized “Tag” complements and the amount of hybridized product is quantitated by the LUMINEX® detector to determine whether normal or mutant sequence has been detected for each mutation of interest.

For a panel consisting of 180 recurrent mutations, PCR amplification of 180 genomic regions containing the 180 mutations to be tested, requiring 360 oligonucleotides as PCR primers, would be carried out on a 16 channel ABI DNA synthesizer. Eight individual multiplex PCR reactions would be instituted with each multiplex containing 18-20 oligonucleotide primer pairs required to amplify the 180 genomic regions containing the mutations of interest.

For a panel consisting of 55 pathogenic mutations, PCR amplification of the genomic regions containing these mutations to be tested with the appropriate number of oligionucleotides as PCR primers would be carried out on a 16 channel ABI DNA synthesizer. Three individual multiplex PCR reactions would be instituted with each multiplex containing 18-20 oligonucleotide primer pairs required to amplify the genomic regions containing the mutations of interest.

One skilled in the art will recognize that the above-described standard methods can be applied to a panel of any number of mutations of interest.

### Validation of the Assay

Samples with known HCM mutations will need to be genotyped to prove that the assay provides accurate results. The Hypertrophic Cardiomyopathy Association can provide access to patient samples that have been sequenced for known HCM mutations and hence can serve as standards to validate the test. These samples also serve as negative controls for all the other mutations in the panel since these mutations were identified by sequencing the genes in which all of the target mutations reside.

### Additional Reference:

Aris, Toruner, Soteropoulos and Dermody. A microarray platform to test the Ashkenazi Jewish population for genetic disease. Microarrays in Medicine (2005), May 4-5, Boston, Mass.

1. A method of screening for hypertrophic cardiomyopathy comprising detecting the presence or absence of at least one pathogenic HCM mutation by mutation detection assay in a sample from a subject to be tested for hypertrophic cardiomyopathy.

2. The method of claim 1, wherein the assay is a particle based allele specific mutation detection assay and wherein:
a. for each HCM mutation to be detected, the assay utilizes one oligonucleotide that matches the mutant DNA sequence and one oligonucleotide that matches the corresponding normal sequence; and
b. each oligonucleotide contains specific sequences that match complementary oligonucleotide sequences on individual detection particles.

3. The method of claim 1, wherein the sample comprises cheek cells.

4. The method of claim 1, wherein the detection is performed by multiplex assay.

5. The method of claim 1, wherein the presence or absence of at least ten pathogenic HCM mutations is detected.

6. The method of claim 1, wherein the presence or absence of at least 55 pathogenic HCM mutations is detected.

7. The method of claim 1, wherein the presence or absence of at least 100 pathogenic HCM mutations is detected.

8. The method of claim 1, wherein the presence or absence of at least 150 pathogenic HCM mutations is detected.

9. The method of claim 1, wherein the detection comprises detecting the presence or absence of from 50 and 600 pathogenic HCM mutations.

10-17. (canceled)

18. The method of claim 1, wherein the catch rate of the method is from 40% to 95%.

19. (canceled)

20. The method of claim 1, wherein the detection is performed by bead based allele specific mutation detection.

21. The method of claim 1, wherein the detection comprises detecting the presence or absence of at least one mutation that is predicted to cause an amino acid substitution and is present in two or more clinically diagnosed HCM patients.

22. The method of claim 1, wherein the detection comprises detecting the presence or absence of at least one mutation whose predicted consequence is the absence of an encoded protein.

23. The method of claim 1, wherein the detection comprises detecting the presence or absence of at least one mutation selected from the group consisting of A6491G, G6643A, T6685C, G8278A, G8848T, G8848A, C8847T, C9123T, A9483G, G10457A, G11282A, G12138A, C12307T, G12361A, delE930, C19222T, AND C19236T in beta-cardiac Myosin Heavy Chain; A5254C, G5256A, G7360A, G11070C, A15829G, G17721A, G20410T, del2376-2381, G5828A, A7308G, A10385G, del10512-10513, delT10587, delC10618, del11047-11048, T11073C, delA12413, A13858G, dup15042-15063, G15131A, A15829G, insG15919, del16189-16193, del16190-16194, delC16212, del17773-17774, del18566-18567, delG21059, ins21404-21415, and del21420-21423 in cardiac Myosin-Binding Protein C; F70L, R102L, P120V, N271I, and W287ter in cardiac Troponin T; and F18L, R58Q, and aIVS5g in cardiac Regulatory Myosin Light Chain.

24. The method of claim 1, wherein the detection comprises detecting the presence or absence of at least 10 mutations selected from the group consisting of A6491G, G6643A, T6685C, G8278A, G8848T, G8848A, C8847T, C9123T, A9483G, G10457A, G11282A, G12138A, C12307T, G12361A, delE930, C19222T, AND C19236T in beta-cardiac Myosin Heavy Chain; A5254C, G5256A, G7360A, G11070C, A15829G, G17721A, G20410T, del2376-2381, G5828A, A7308G, A10385G, del10512-10513, delT10587, delC10618, del11047-11048, T11073C, delA12413, A13858G, dup15042-15063, G15131A, A15829G, insG15919, del16189-16193, del16190-16194, delC16212, del17773-17774, del18566-18567, delG21059, ins21404-21415, and del21420-21423 in cardiac Myosin-Binding Protein C; F70L, R102L, P120V, N2711, and W287ter in cardiac Troponin T; and F18L, R58Q, and aIVS5g in cardiac Regulatory Myosin Light Chain.

25. (canceled)

26. The method of claim 1, wherein the detection comprises detecting the presence or absence of at least 30 mutations selected from the group consisting of A6491G, G6643A, T6685C, G8278A, G8848T, G8848A, C8847T, C9123T, A9483G, G10457A, G11282A, G12138A, C12307T, G12361A, delE930, C19222T, AND C19236T in beta-cardiac Myosin Heavy Chain; A5254C, G5256A, G7360A, G11070C, A15829G, G17721A, G20410T, del2376-2381, G5828A, A7308G, A10385G, del10512-10513, delT10587, delC10618, del11047-11048, T11073C, delA12413, A13858G, dup15042-15063, G15131A, A15829G, insG15919, del16189-16193, del16190-16194, delC16212, del17773-17774, del18566-18567, delG21059, ins21404-21415, and del21420-21423 in cardiac Myosin-Binding Protein C; F70L, R102L, P120V, N271I, and W287ter in cardiac Troponin T; and F18L, R58Q, and aIVS5g in cardiac Regulatory Myosin Light Chain.

27. (canceled)

28. The method of claim 1, wherein the detection comprises detecting the presence or absence of at least 50 mutations selected from the group consisting of A6491G, G6643A, T6685C, G8278A, G8848T, G8848A, C8847T, C9123T, A9483G, G10457A, G11282A, G12138A, C12307T, G12361A, delE930, C19222T, AND C19236T in beta-cardiac Myosin Heavy Chain; A5254C, G5256A, G7360A, G11070C, A15829G, G17721A, G20410T, del2376-2381, G5828A, A7308G, A10385G, del10512-10513, delT10587, delC10618, del11047-11048, T11073C, delA12413, A13858G, dup15042-15063, G15131A, A15829G, insG15919, del16189-16193, del16190-16194, delC16212, del17773-17774, del18566-18567, delG21059, ins21404-21415, and del21420-21423 in cardiac Myosin-Binding Protein C; F70L, R102L, P120V, N271I, and W287ter in cardiac Troponin T; and F18L, R58Q, and aIVS5g in cardiac Regulatory Myosin Light Chain.

29. A method of diagnosing hypertrophic cardiomyopathy comprising detecting the presence of at least one pathogenic HCM mutation by mutation detection assay in a sample from a subject to be tested for hypertrophic cardiomyopathy.

30. A method of diagnosing hypertrophic cardiomyopathy comprising detecting the presence of at least one pathogenic HCM mutation by a particle based allele specific mutation detection assay in a sample from a subject to be tested for hypertrophic cardiomyopathy, wherein:
a. for each HCM mutation to be detected, the assay utilizes one oligonucleotide that matches the mutant DNA sequence and one oligonucleotide that matches the corresponding normal sequence; and
b. each oligonucleotide contains specific sequences that match complementary oligonucleotide sequences on individual detection particles.

31. A diagnostic apparatus comprising a mutation detection system capable of detecting the presence or absence of at least one pathogenic HCM mutation in a sample from a subject to be tested for hypertrophic cardiomyopathy.

32. A diagnostic apparatus comprising a mutation detection system capable of detecting the presence or absence of at least one pathogenic HCM mutation by particle based allele specific mutation detection assay in a sample from a subject to be tested for hypertrophic cardiomyopathy, wherein:
a. for each HCM mutation to be detected, the assay utilizes one oligonucleotide that matches the mutant DNA sequence and one oligonucleotide that matches the corresponding normal sequence; and
b. each oligonucleotide contains specific sequences that match complementary oligonucleotide sequences on individual detection particles.
